(19) United States
(12) Patent Application Publication
(10) Pub. No.: US 2019/0274575 A1
(43) Pub. Date: Sep. 12, 2019

(54) METHOD AND APPARATUS FOR DIAGNOSING LUMBAR SPINAL STENOSIS

(71) Applicant: KOREA UNIVERSITY RESEARCH AND BUSINESS FOUNDATION, Seoul (KR)

(72) Inventor: Dong-Joo KIM, Seoul (KR)

(21) Appl. No.: 16/191,985

(22) Filed: Nov. 15, 2018

(30) Foreign Application Priority Data
Mar. 7, 2018 (KR) 10-2018-0026899

(51) Int. Cl. A61B 5/055; A61B 5/00; G01R 33/28; G06T 7/00

(52) U.S. Cl. CPC A61B 5/055; A61B 5/0042; G01R 33/56308; G06T 7/0012; G01R 33/281

(57) ABSTRACT

The present disclosure relates to a spinal stenosis diagnostic method and apparatus and the spinal stenosis diagnostic method includes: sequentially receiving a phase contrast magnetic resonance imaging in each time interval captured by normalizing one cardiac cycle with a plurality of time intervals; obtaining a cerebrospinal fluid velocity distribution in each normalized time interval from the phase contrast magnetic resonance imaging of each time interval; calculating a turbulence kinetic energy using the cerebrospinal fluid velocity distribution obtained at every time interval; and diagnosing the spinal stenosis using the calculation result of the turbulence kinetic energy.

### CROSS-REFERENCE TO RELATED APPLICATIONS

This application claims the priority of Korean Patent Application No. 10-2018-0026899 filed on Mar. 7, 2018, in the Korean Intellectual Property Office, the disclosure of which is incorporated herein by reference.

### BACKGROUND

### Field

The present disclosure relates to a diagnosing method using a spinal phase contrast magnetic resonance imaging and more particularly, to a method for diagnosing a lumbar spinal stenosis using a phase contrast magnetic resonance imaging of a cerebrospinal fluid.

### Description of the Related Art

A spinal canal refers to a space enclosed by a centrum which is an anterior part f the vertebra and the intervertebral disc (disc) at the front side and a lamina of vertebral arch which is a posterior part of the vertebra at the rear side. The spinal canal is a pathway of nerves which start from the brain and pass through the cervical vertebra (neck bone) and the thoracic vertebra (backbone) and are continued from the lumbar vertebra (waist) to nether extremities (hip, legs, and feet).

The spinal stenosis is a state that the spinal canal through which the nerves pass is narrowed. When the disk is damaged, the spinal stenosis is caused. The intervertebral disc is a disc of cartilage interposed between the vertebrae of the spine and has elasticity to provide the flexibility to the backbone and serves as a cushion which absorbs the impact. When an excessive impact is applied, the intervertebral disc is ruptured and slipped out. When the people become elderly and lack resilience, it is especially susceptible. In this case, a state in which the intervertebral disc is slipped out is called a spinal disc. In this case, the slipped intervertebral disc compresses the spinal cord which is the central nerve to cause pains, paralysis, or dysfunction of the entire body.

The symptoms of the spinal stenosis include lumbar pain, nervous intermittent claudication, lower radiation pain, lower extremity sensory abnormality, neck pain, upper extremity pain, arm motion and sensory abnormalities, and myelopathy. In order to diagnose the symptoms of spinal stenosis, a method of analyzing the magnetic resonance imaging (MRI) of the vertebra is used.

The magnet resonance imaging of the vertebra is the primary tool for diagnosing the patients with the spinal stenosis and is being mainly used to diagnose the spinal stenosis. However, the spinal stenosis by the magnetic resonance imaging does not result in the diagnosis of the actual disease. Even though it is analyzed that the spinal stenosis is discovered from the magnetic resonance imaging, a subject actually does not feel pain so that misdiagnosis may occur occasionally.

In the case of the spinal stenosis, it has low specificity of the diagnosis because even though the stenosis is discovered from the magnetic resonance imaging analysis, the patient is normal in some cases. Therefore, a new method for diagnosing the spinal stenosis is needed.

### SUMMARY

An object of the present disclosure is to provide a method and an apparatus for diagnosing a spinal stenosis to diagnose a lumbar spinal stenosis by calculating turbulence kinetic energy of the cerebrospinal fluid from the spinal phase contrast magnetic resonance imaging.

According to an aspect of the present disclosure, a spinal stenosis diagnostic method includes: sequentially receiving a phase contrast magnetic resonance imaging in each time interval captured by normalizing one cardiac cycle with a plurality of time intervals, obtaining a cerebrospinal fluid velocity distribution in each normalized time interval from the phase contrast magnetic resonance imaging of each time interval, calculating a turbulence kinetic energy using the cerebrospinal fluid velocity distribution obtained at every time interval, and diagnosing the spinal stenosis using the calculation result of the turbulence kinetic energy.

Further, the spinal stenosis diagnostic method may further include: between the receiving of the phase contrast magnetic resonance imaging and the obtaining of the cerebrospinal fluid velocity distribution of the normalized time interval, setting a region of interest (ROI) having a predetermined size in the phase contrast magnetic resonance imaging in each time interval.

Further, the obtaining of the cerebrospinal fluid velocity distribution may include calculating a mean value of pixel values for the ROI by applying each pixel value of the ROI to the following equation and calculating the mean velocity of the cerebrospinal fluid using at least one of a mean value of the calculated pixel values, a mean value of a magnetic resonance pixel value of a size of a machine which captures the phase contrast magnetic resonance imaging, and an encoding velocity.

$${MP}_{i} = \frac{{\sum }_{j = 1}^{n} C ( {P}_{i - j} )}{n} .$$

(Here, MPi is a mean value of a pixel value in an ROI of an i-th slide, Pi, j is a value of a j-th pixel in an ROI of the phase contrast magnetic resonance imaging of the i-th slide, and C(Pi, j) is a result obtained by applying a calibration function written in an image header to the pixel value)

Further, in the calculating of the turbulence kinetic energy (CSF TKE), the turbulence kinetic energy may be calculated by the following Equation using the velocity distribution of the cerebrospinal fluid and the mean velocity of the cerebrospinal fluid.

$$CSF TKE = \frac{1}{2 T} {\int }_{i = 0}^{n} {( CSFv ( {t}_{i} ) - {CSFv}_{mean} )}^{2} dt$$

(Here, T denotes a cardiac cycle and i denotes each time interval obtained by dividing a cardiac cycle by n)

In the diagnosing of the spinal stenosis, when the turbulence kinetic energy is equal to or lower than 0.0678 cm²/s·kg, a subject may be diagnosed as a patient group.

According to another aspect of the present disclosure, a computer readable recording medium in which a program which implements the spinal stenosis diagnostic method is recorded is provided.

According to still another aspect of the present disclosure, a spinal stenosis diagnostic apparatus includes an input unit which sequentially receives a phase contrast magnetic resonance imaging in each time interval captured by normalizing one cardiac cycle with a plurality of time intervals and a control unit which obtains a cerebrospinal fluid velocity distribution in each normalized time interval from the phase contrast magnetic resonance imaging of each time interval received through the input unit, calculates a turbulence kinetic energy using the cerebrospinal fluid velocity distributionobtainedateverytimeinterval,anddiagnosesthespinal stenosis using the calculation result of the turbulence kinetic energy.

According to the present disclosure, the spinal stenosis diagnosis may be automated and the accuracy may be improved by automatically calculatingtheobjectifiedturbulencekineticenergy from the phase contrast magnetic resonance imaging.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a schematic flowchart of a spinal stenosis diagnostic method according to an exemplary embodiment of the present disclosure;

FIG. 2 is a schematic diagram of a spinal stenosis diagnostic apparatus according to an exemplary embodiment of the present disclosure;

FIG. 3A to FIG. 3L are views for explaining a spinal stenosis diagnostic method according to an exemplary embodiment of the present disclosure by comparing the phase contrast magnetic resonance imaging and a velocity of a cerebrospinal fluid;

FIG. 4A and FIG. 4B are views for explaining a spinal stenosis diagnostic method according to an exemplary embodiment of the present disclosure by comparing velocities of cerebrospinal fluid of a healthy people group and a patient group; and

FIGS. 5A to FIG. 5D, FIG. 6A to FIG. 6C, FIG. 7A to FIG. 7C are views for determining major parameters of a spinal stenosis diagnostic method according to an exemplary embodiment of the present disclosure.

### DETAILED DESCRIPTION OF THE PREFERRED EMBODIMENT

Those skilled in the art may make various modifications to the present invention and the present invention may have various embodiments thereof, and thus specific embodiments will be described in detail with reference to the drawings. However, this does not limit the present invention within specific exemplary embodiments, and it should be understood that the present invention covers all the modifications, equivalents and replacements within the spirit and technical scope of the present invention. In the description of respective drawings, similar reference numerals designate similar elements.

Terms such as first, second, A, or B may be used to describe various components but the components are not limited by the above terms. The above terms are used only to discriminate one component from the other component. For example, without departing from the scope of the present invention, a first component may be referred to as a second component, and similarly, a second component may be referred to as a first component. A term of and/or includes combination of a plurality of related elements or any one of the plurality of related elements.

It should be understood that, when it is described that an element is “coupled” or “connected” to another element, the element may be directly coupled or directly connected to the other element or coupled or connected to the other element through a third element. In contrast, when it is described that an element is “directly coupled” or “directly connected” to another element, it should be understood that no element is present therebetween.

Terms used in the present application are used only to describe a specific exemplary embodiment, but are not intended to limit the present invention. A singular form may include a plural form if there is no clearly opposite meaning in the context. In the present invention, it should be understood that terminology “include” or “have” indicates that a feature, a number, a step, an operation, a component, apart or the combination thoseof described in the specification is present, but does not exclude a possibility of presence or addition of one or more other features, numbers, steps, operations, components, parts or combinations, in advance.

If it is not contrarily defined, all terms used herein including technological or scientific terms have the same meaning as those generally understood by a person with ordinary skill in the art. Terms defined in generally used dictionary shall be construed that they have meanings matching those in the context of a related art, and shall not be construed in ideal or excessively formal meanings unless they are clearly defined in the present application.

In the specification and the claim, unless explicitly described to the contrary, the word “comprise” and variations such as “comprises” or “comprising,” will be understood to imply the inclusion of stated elements but not the exclusion of any other elements.

Hereinafter, exemplary embodiments according to the present disclosure will be described in detail with reference to accompanying drawings.

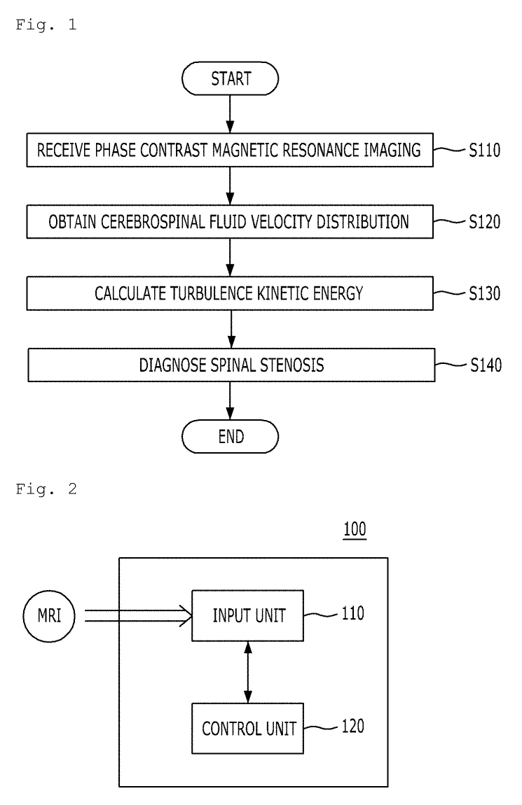

FIG. 1 is a schematic flowchart of a spinal stenosis diagnostic method according to an exemplary embodiment of the present disclosure.

Referring to FIG. 1, a spinal stenosis diagnostic method according to an exemplary embodiment of the present disclosure may be performed in the order of a step S110 of receiving a phase contrast magnetic resonance imaging, a step S120 of obtaining velocity distribution of a cerebrospinal fluid, a step S130 of calculating a turbulence kinetic energy, and a step S140 of diagnosing a spinal stenosis.

First, in the step of receiving a phase contrast magnetic resonance imaging, the phase contrast magnetic resonance imaging captured by an external phase contrast magnetic resonance imaging (PC-MRI) device may be received. The PC-MRI device normalizes one cardiac cycle with a plurality of time intervals to capture a phase contrast magnetic resonance imaging of each time interval.

Specifically, the cardiac cycle refers to a period during which the ventricle is contracted while the atrium is contracted and relaxed and the ventricle and the atrium are relaxed together and then the atrium is contracted. The spinal stenosis is a disease which causes pains of lumbar vertebra or sacrum portions and relates to the phase contrast magnetic resonance imaging of the lumbar vertebra or sacrum portions. Specifically, when the spinal stenosis is diagnosed using a flow velocity of the cerebrospinal fluid, since the correlation between the flow velocity of the cerebrospinal fluid of the lumbar vertebra portion and the disease is high, the phase contrast magnetic resonance imaging of this portion is used.

A region of interest (ROI) having a predetermined size is set in the phase contrast magnetic resonance imaging of the lumbar vertebra captured in a vertical direction to a vertebra connection line and the turbulence kinetic energy is calculated using the flow velocity distribution of the cerebrospinal fluid for the ROI. For example, the ROI may be selected to have a square shape having a size of 0.3 cm×0.3 cm and include a plurality of pixel values in the corresponding region. In this case, parameters in accordance with the characteristics of the PC-MRI device capturing the phase contrast magnetic resonance imaging, such as a thickness of the captured slide or a velocity encoding parameter may be determined.

Next, in the step S120 of obtaining the distribution of the velocity of a cerebrospinal fluid, a phase contrast magnetic resonance imaging sequentially received in each time interval, specifically, an image of the ROI of the phase contrast magnetic resonance imaging is analyzed to obtain the velocity distribution of the cerebrospinal fluid. Specifically, the obtained phase contrast magnetic resonance imaging is captured by normalizing one cardiac cycle with N time intervals. The time of the cardiac cycle varies depending on the subject, so that it is necessary to normalize one cardiac cycle with the N time intervals to normalize a flow velocity and determine the velocity distribution of the cerebrospinal fluid using a displacement value in one direction of the normalized flow velocity in each time interval.

The velocity distribution of the cerebrospinal fluid is determined so that a mean value of pixel values for the ROI may be calculated by applying each pixel value of the ROI to Equation 1.

$${MP}_{i} = \frac{{\sum }_{j = 1}^{n} C ( {P}_{i - j} )}{n} . \qquad Equation 1$$

Here, MPi is a mean value of pixel values in an ROI of an i-th slide, Pi, j is a value of a j-th pixel in an ROI of the phase contrast magnetic resonance imaging of the i-th slide, and C(Pi, j) is a result obtained by applying a calibration function written in an image header to the pixel value.

The velocity distribution of the cerebrospinal fluid may be calculated using at least one of the mean value of pixel values, MPi, calculated by Equation 1, a mean value of an amplitude MRI pixel value, MAi, which captures the phase contrast magnetic resonance imaging, MAi, and an encoding velocity (VENC).

For example, when a GE machine is used, the velocity distribution of the cerebrospinal fluid is calculated by Equation 2 and when Siemens and Philips machines are used, the velocity distribution of the cerebrospinal fluid is calculated by Equation 3.

$$\begin{matrix}Mean flow velocity ( mm \text{/} sec ) = \frac{{MP}_{i} + {MA}_{i}}{2} \times \frac{VENC}{scale value \times π} & Equation 2 \\ Mean flow velocity ( mm \text{/} sec ) = \frac{{MP}_{i} - 2048}{4096} \times 2 \times VENC & Equation 3\end{matrix}$$

Next, in the step S130 of calculating a turbulence kinetic energy CSF TKE, the turbulence kinetic energy may be calculated by Equation 4 using the velocity distribution of the cerebrospinal fluid and the mean velocity of the cerebrospinal fluid.

$$CSF TKE = \frac{1}{2 T} {\int }_{i = 0}^{n} {( CSFv ( {t}_{i} ) - {CSFv}_{mean} )}^{2} dt \qquad Equation 4$$

Here, T denotes a cardiac cycle and i denotes each time interval obtained by dividing a cardiac cycle by n.

Finally, in the step S140 of diagnosing the spinal stenosis, when the turbulence kinetic energy exceeds a reference value, the subject is diagnosed as a healthy people group and when the turbulence kinetic energy is equal to or lower than the reference value, the subject is diagnosed as a patient group. Specifically, when the turbulence kinetic energy is equal to or lower than 0.0678 cm2/s·kg, the subject is diagnosed as a patient group.

FIG. 2 is a schematic diagram of the spinal stenosis diagnostic apparatus according to an exemplary embodiment of the present disclosure.

Referringto FIG. 2, the spinal stenosis diagnostic apparatus 100 according to an exemplary embodiment of the present disclosure includes an input unit 110 and a control unit 120.

The input unit 110 sequentially receives the phase contrast magnetic resonance imaging in each time interval captured by normalizing one cardiac cycle with a plurality of time intervals. The input unit 110 receives the phase contrast magnetic resonance imaging captured by the external PC-MRI device and the PC-MRI device captures the images by normalizing one cardiac cycle with a plurality of time intervals. The control unit 120 obtains the cerebrospinal fluid velocity distribution from the phase contrast magnetic resonance imaging received through the input unit 110, calculates the turbulence kinetic energy using the cerebrospinal fluid velocity distribution obtained for every time interval, and diagnoses the spinal stenosis using the calculation result of the turbulence kinetic energy. Specific functions of the control unit 120 have been described above with reference to FIG. 1 so that a specific description will be omitted.

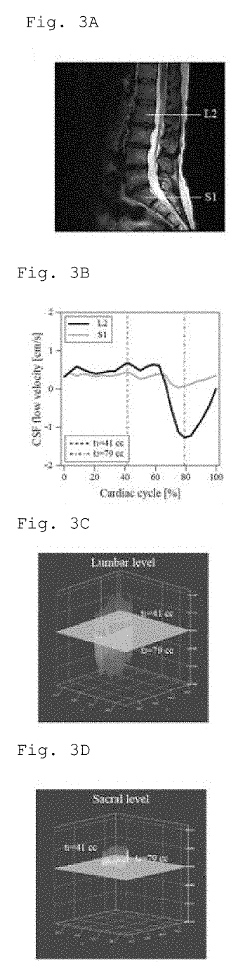

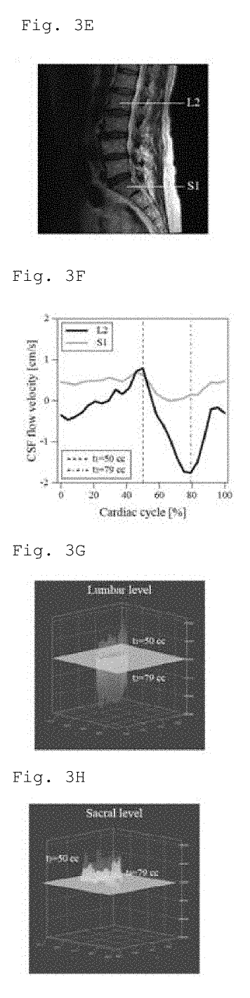

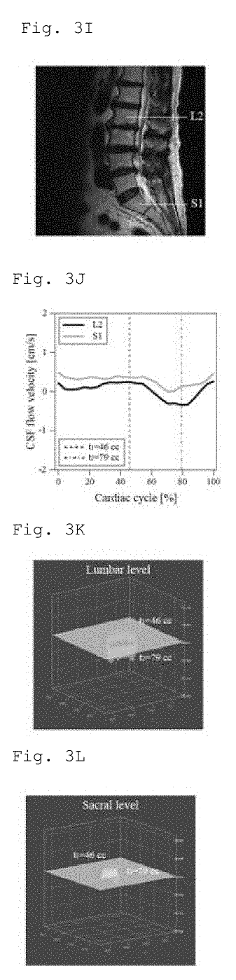

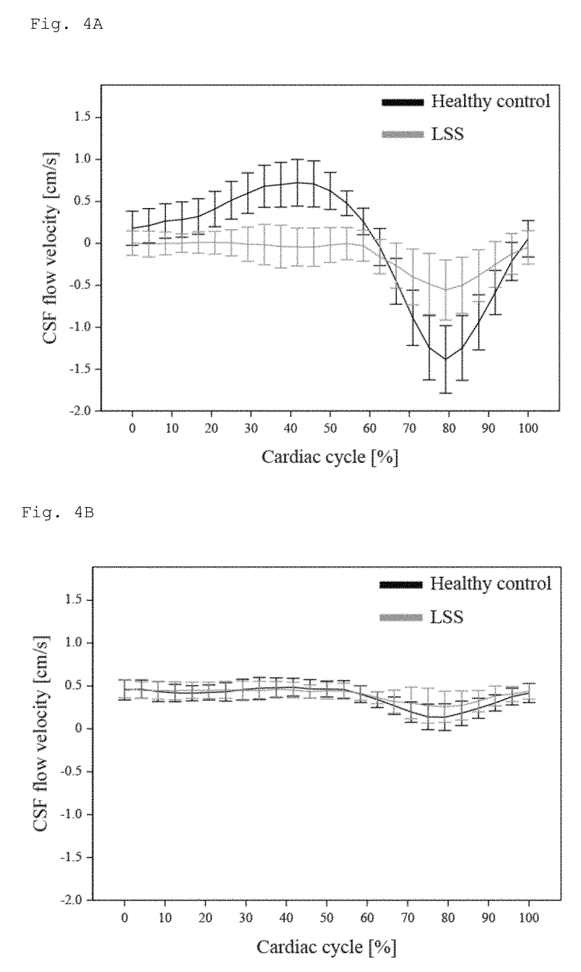

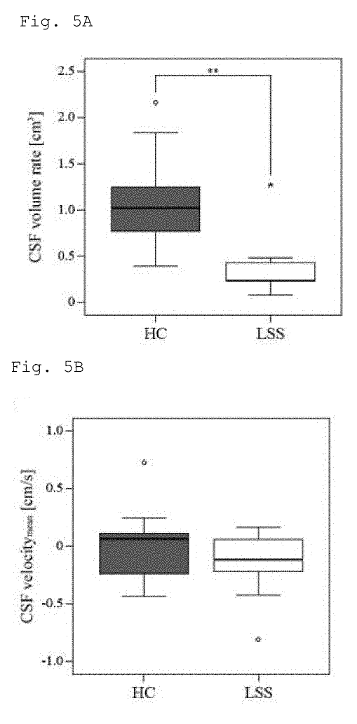

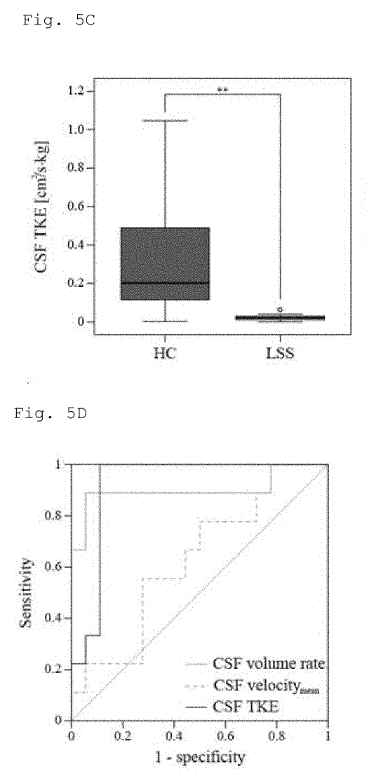

FIG. 3A to FIG. 3L are views for explaining a spinal stenosis diagnostic method according to an exemplary embodiment of the present disclosure by comparing the phase contrast magnetic resonance imaging and a velocity of cerebrospinal fluid and FIG. 4A and FIG. 4B are views for explaining a spinal stenosis diagnostic method according to an exemplary embodiment of the present disclosure by comparing velocities of a cerebrospinal fluid of a healthy people group and a patient group.

Referring to FIG. 3A to FIG. 3L, FIG. 3A to FIG. 3D show a phase contrast magnetic resonance imaging of a subject in a healthy people group who has a wide spinal canal and does not exhibit the symptom, FIG. 3E to FIG. 3H show a phase contrast magnetic resonance imaging of a subject in a healthy people group who has a narrow spinal canal, but does not exhibit the symptom, and FIG. 3I to FIG. 3L show a phase contrast magnetic resonance imaging of a subject in a patient group who has a narrow spinal canal and exhibits the symptom.

The spinal stenosis is suspicious only from the phase contrast magnetic resonance imaging in the case of FIG. 3E to FIG. 3H. However, even though the spinal canal is narrow, in many cases, the symptom does not occur. Therefore, there is a risk of misdiagnosis when only the phase contrast magnetic resonance imaging is analyzed.

In the cases of FIG. 3A to FIG. 3L, when the flow velocities of the cerebrospinal fluid in the lumbar vertebra L2 and the sacrum S1 are measured, patterns as illustrated in second graphs may be obtained. That is, there is no significant difference of the flow velocity of the cerebrospinal fluid in the sacrum S1 between the patient group and the healthy people group, but there is a difference of the flow velocity of the cerebrospinal fluid in the lumbar vertebra L2. It is understood that when the flow velocity of the cerebrospinal fluid in the lumbar vertebra L2 shows a similar pattern to that of the subject of the healthy people group, that is, in the case of B with the large flow velocity change, the subject has a narrow spinal canal, but does not exhibit the symptom of the spinal stenosis.

Referring to FIG. 4A and FIG. 4B, it is confirmed that as compared with the healthy people group, the cerebrospinal fluid flow velocity of the patient group has a big difference in the lumbar vertebra.

When the velocity displacement in one direction with respect to a time axis is detected from the phase contrast magnetic resonance imaging, the size of the y-axis displacement of the patient group is smaller than that of the healthy people group.

Specifically, the difference is significant in the interval of 20 to 54% (p<0.001) of one cardiac cycle. Therefore, it is confirmed that the cerebrospinal fluid velocity of the patient with the spinal stenosis is lower than that of the healthy people group and the difference is more significant in a portion which flows downwardly during one cycle. When the velocity of the cerebrospinal fluid is converted into the turbulence kinetic energy, the difference is more significant.

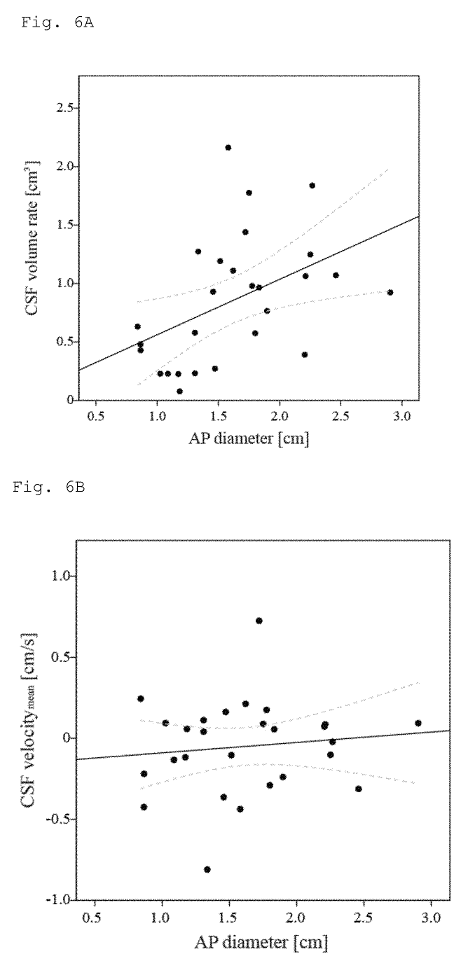

FIGS. 5A to FIG. 5D, FIG. 6A to FIG. 6C, and FIG. 7A to FIG. 7C are views for determining major parameters of a spinal stenosis diagnostic method according to an exemplary embodiment of the present disclosure.

Referring to FIG. 5A to FIG. 5D, when the relationships of a volume rate (CSF volume rate) of the cerebrospinal fluid, a velocity (CSF velocity) of the cerebrospinal fluid, and a turbulence kinetic energy (CSF TKE) of the cerebrospinal fluid of the healthy people group and the patient group are compared, it is confirmed that the biggest difference for distinguishing the patient group and the healthy people group is the turbulence kinetic energy (CSF TKE) of the cerebrospinal fluid.

Referring to FIG. 6A to FIG. 6C, when the relationships of a volume rate (CSF volume rate) of the cerebrospinal fluid, avelocity (CSF velocity) of the cerebrospinal fluid, and a turbulence kinetic energy (CSF TKE) in accordance with a sum of diameters of the spinal canal in each spinal layer, that is, the degree of stenosis are checked, it is confirmed that the velocity (CSF velocity) of the cerebrospinal fluid and the turbulence kinetic energy (CSF TKE) of the cerebrospinal fluid are not correlated with the degree of stenosis.

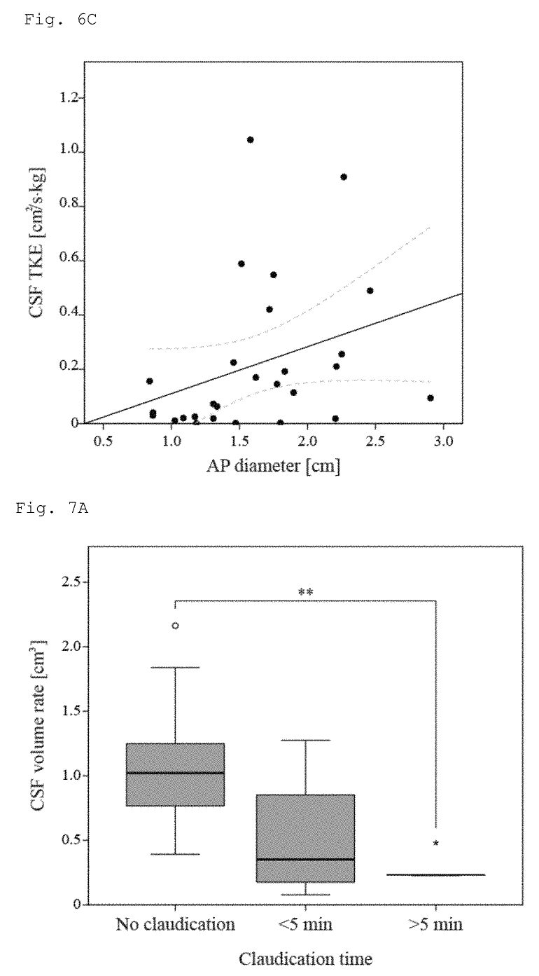

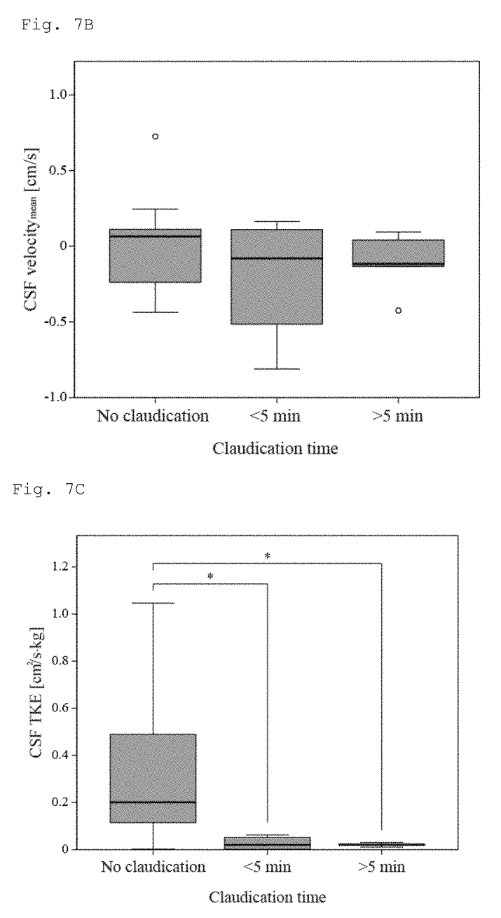

Referring to FIG. 7A to FIG. 7C, it is confirmed that a claudication time, that is, the severity of the symptom and some dynamic parameters of the cerebrospinal fluid have a significant relationship. Even though the mean CSF velocity does not have a significant statistical difference according to the severity of the symptom, the CSF volume rate and the CSF TKE have higher values when the severity of the patient group is low. Therefore, it is confirmed that the CSF TKE among hemodynamic parameters of the cerebrospinal fluid is not correlated with the degree of stenosis, but varies depending on the severity of the symptom.

According to the present disclosure, the velocity and the turbulence kinetic energy of the cerebrospinal fluid are calculated from the phase contrast magnetic resonance imaging to objectify the spinal stenosis diagnosis and improve the accuracy.

The spinal stenosis diagnostic method according to the exemplary embodiment of the present disclosure described above may be executed by an application (which may include programs included in a platform or an operating system basically mounted in a terminal) which is basically installed in the spinal stenosis diagnostic apparatus or executed by an application (program) which is directly installed in a terminal by means of an application providing server such as an application store server or a web server related to the application or the service, by a user.

In this regard, the spinal stenosis diagnostic apparatus according to the exemplary embodiment of the present disclosure described above may be implemented by an application (program) which is basically installed in a terminal or directly installed by the user and recorded in a recording mediumwhich is readable by a computer such as a terminal . Such a program is recorded in a computer readable recording medium and executed by the computer to execute the above-mentioned functions.

As described above, the program for executing the spinal stenosis diagnostic method according to the exemplary embodiment of the present disclosure may include a code encoded into a computer language such as C, C++, JAVA, or a machine language which can be read by a processor (CPU) of the computer.

Such a code may further include a memory reference related code indicating a location (address) of an internal or external memory of the computer where additional information or media required to allow the processor of the computer to execute the above-mentioned functions is referenced.

A functional program for implementing the present disclosure and a code and a code segment related thereto may be easily inferred or modified by programmers in the technical field of the present disclosure in consideration of a system environment of a computer which reads the recording medium to execute the program.

The computer readable recording medium in which the program is recorded may include ROM, RAM, CD-ROM, a magnetic tape, a floppy disk, and an optical media storage device.

Even though all components of the exemplary embodiment of the present invention may be combined as one component or operates to be combined, the present invention is not limited to the exemplary embodiment. In other words, all components may be selectively combined as at least one to be operated within a scope of the present invention. Further, all of the components may be implemented as one independent hardware but a part or all of the components are selectively combined to be implemented as a computer program which includes a program module which performs a part or all of functions combined in one or plural hardwares. Further, codes and code segments which configure the computer program may be easily deduced by those skilled in the art. The computer program is stored in computer readable media to be read and executed by a computer so as to implement an exemplary embodiment of the present invention. The recording media of the computer program may include a magnetic recording medium or an optical recording medium.

It will be appreciated that various exemplary embodiments of the present invention have been described herein for purposes of illustration, and that various modifications, changes, and substitutions may be made by those skilled in the art without departing from the scope and spirit of the present invention. Therefore, the exemplary embodiments of the present disclosure are provided for illustrative purposes only but not intended to limit the technical concept of the present disclosure. The scope of the technical concept of the present disclosure is not limited thereto. The protection scope of the present invention should be interpreted based on the following appended claims and it should be appreciated that all technical spirits included within a range equivalent thereto are included in the protection scope of the present invention.

What is claimed is:

1. A spinal stenosis diagnostic method, comprising:
sequentially receiving a phase contrast magnetic resonance imaging in each time interval captured by normalizing one cardiac cycle with a plurality of time intervals;
obtaining a cerebrospinal fluid velocity distribution in each normalized time interval from the phase contrast magnetic resonance imaging of each time interval;
calculating a turbulence kinetic energy using the cerebrospinal fluid velocity distribution obtained at every time interval; and
diagnosing the spinal stenosis using the calculation result of the turbulence kinetic energy.

2. The spinal stenosis diagnostic method according to claim 1, further comprising:
between the receiving of the phase contrast magnetic resonance imaging and the obtaining of the cerebrospinal fluid velocity distribution of the normalized time interval,
setting a region of interest (ROI) having a predetermined size in the phase contrast magnetic resonance imaging in each time interval.

3. The spinal stenosis diagnostic method according to claim 2, wherein the obtaining of the cerebrospinal fluid velocity distribution includes:
calculating a mean value of pixel values for the ROI by applying each pixel value of the ROI to the following equation; and
calculating the mean velocity of the cerebrospinal fluid using at least one of a mean value of the calculated pixel values, a mean value of a magnetic resonance pixel value of a size of a machine which captures the phase contrast magnetic resonance imaging, and an encoding velocity.
$${MP}_{i} = \frac{{\sum }_{j = 1}^{n} C ( {P}_{i - j} )}{n} .$$
(Here, MPi is a mean value of a pixel value in an ROI of an i-th slide, Pi, j is a value of a j-th pixel in an ROI of the phase contrast magnetic resonance imaging of the i-th slide, and C(Pi, j) is a result obtained by applying a calibration function written in an image header to the pixel value)

4. The spinal stenosis diagnostic method according to claim 3, wherein in the calculating of the turbulence kinetic energy (CSF TKE), the turbulence kinetic energy is calculated by the following Equation using the velocity distribution of the cerebrospinal fluid and the mean velocity of the cerebrospinal fluid.
$$CSF TKE = \frac{1}{2 T} {\int }_{i = 0}^{n} {( CSFv ( {t}_{i} ) - {CSFv}_{mean} )}^{2} dt$$
(Here, T denotes a cardiac cycle and i denotes each time interval obtained by dividing a cardiac cycle by n)

5. The spinal stenosis diagnostic method according to claim 4, wherein in the diagnosing of the spinal stenosis, when the turbulence kinetic energy is equal to or lower than 0.0678 cm2/s·kg, a subject is diagnosed as a patient group.

6. A computer readable recording medium in which a program which implements the spinal stenosis diagnostic method of claim 1 is recorded.

7. A spinal stenosis diagnostic apparatus, comprising:
an input unit which sequentially receives a phase contrast magnetic resonance imaging in each time interval captured by normalizing one cardiac cycle with a plurality of time intervals; and
a control unit which obtains a cerebrospinal fluid velocity distribution in each normalized time interval from the phase contrast magnetic resonance imaging of each time interval received through the input unit, calculates a turbulence kinetic energy using the cerebrospinal fluid velocity distribution obtained at every time interval, and diagnoses the spinal stenosis using the calculation result of the turbulence kinetic energy.
